(19) United States
(12) Patent Application Publication
(10) Pub. No.: US 2010/0015164 A1
(43) Pub. Date: Jan. 21, 2010

(54) Compositions and Methods for Modulating Osteoblast Cell Differentiation and Bone Generation Through HIF-1a

(75) Inventors: Thomas L. Clemens, Birmingham, AL (US); Randall S. Johnson, San Diego, CA (US); Ernestina Schipani, Boston, MA (US)

Correspondence Address: SUTHERLAND ASBILL & BRENNAN LLP, 999 PEACHTREE STREET, N.E., ATLANTA, GA, 30309, US

(73) Assignee: THE UAB RESEARCH FOUNDATION

(21) Appl. No.: 12/096,733

(22) PCT Filed: Dec. 11, 2006
(86) PCT No.: PCT/US06/61883

§ 371 Date: Jul. 7, 2009

Related U.S. Application Data
(60) Provisional application No. 60/749,275, filed on Dec. 9, 2005.

(51) Int. Cl. A61K 39/395; C12Q 1/02; C12Q 1/68

(52) U.S. Cl. 424/178.1; 435/29; 435/6

(57) ABSTRACT

The present invention provides a novel screening tool for the determination of compounds capable of promoting bone healing, promoting osteoblast cellular differentiation, improving bone mass or volume, and/or promoting osteogenesis that could be used in the treatment of various bone-loss or bone density decreasing disorders. The present invention also provides a screening tool for the determination of compounds capable of inhibiting osteoblast cellular differentiation, decreasing bone mass or volume, and/or inhibiting osteogenesis. Also provided are compositions and methods for the treatment of various bone-loss or bone density decreasing disorders.

### CROSS REFERENCES TO RELATED APPLICATIONS

This application claims the priority benefit of U.S. Provisional Application No. 60/749,275, filed Dec. 9, 2005, and the contents of which are hereby incorporated in their entirety.

### ACKNOWLEDGEMENT OF FEDERAL RESEARCH SUPPORT

This invention was made, at least in part, with funding from the National Institutes of Health (Award Number 1R01 AR049410-0141). Accordingly, the United States Government has certain rights in this invention.

### BACKGROUND OF THE INVENTION

1. Field of the Invention

The present invention relates to the discovery of a novel pathway for the induction of osteoblast cellular differentiation and bone generation. Specifically, the present invention envisions a novel screening tool for the determination of compounds capable of promoting osteoblast cellular differentiation, improving bone mass or volume, and/or promoting osteogenesis that could be used in the treatment of various bone-loss or bone density decreasing disorders.

2. Background

Impaired bone healing after trauma, surgery, and/or infections can be a difficult clinical problem. Distraction osteogenesis (DO) is a powerful tool in the management of bone loss, for correction of deformity and repair of defects secondary to trauma and/or infection. The concept of distraction osteogenesis was developed by the Russian surgeon Ilizarov in the 1950's, made its way to western medicine in the 1980's, and is widely applied today. It is a dramatic display of the body's ability to produce bone and surrounding tissues in response to mechanical stimulation. However, despite intensive investigation, the mechanisms underlying the new bone formation observed are not well understood. In general, bone is formed directly by intramembranous (osteoblast-mediated with this technique) ossification without an endochondral platform. Following surgical corticotomy there is a latency period prior to distraction that is characterized by an inflammatory response like that following routine fracture. As the bone is distracted, a migrating zone of proliferating, fibroblast-tike cells appear and align parallel to the vector of elongation. Collagen is formed in bundles and capillaries form between them. Osteoblasts then arrange themselves along the collagen framework and lay down woven bone which is subsequently remodeled into lamellar bone during the consolidation phase after distraction is halted. Angiogenesis is a striking feature and critical component of the process of distraction histiogenesis. New blood vessels have been shown to be oriented parallel to the direction of distraction where new bone is forming along parallel collagen bundles, implying a coupling of the mechanical force applied and neovascularization.

In clinical practice, delays in ossification are not uncommon and relate to inappropriate application of the method (inadequate fixation, improper rate of distraction) or unfavorable host factors such as a poor soft tissue envelope and/or compromised vascularity. In a murine model, Choi et al. were able to induce non-union of a distraction gap by increasing the rate of distraction from their standard of 0.24 mm/day by 300% to 0.72 mm/day and eliminating the latency period (2004, J. Orthop. Res. 22:1100-7). Another investigator noted that 0.84 mm/day distraction resulted in inadequate bone formation (Tay et al., 1998, J. Orthop. Res. 16:636-42), whereas most murine models have distracted at a rate of 0.15 to 0.3 mm/day with reliable bone healing (Carvalho et al., 2004, Bone 34:849-61; Choi et al., 2004, J. Orthop. Res. 22:1100-7; Fang et al., 2004, Bone 34:1004-12; Isefuku et al, 2000, Bone 27:661-65). In the non-union model above, the authors noted that the distraction gap at the conclusion of distraction contained a large avascular zone with absent PECAM-1 staining. The gap eventually vascularized, but non-union persisted. Therefore, vascularization is necessary, but not sufficient for bone formation in DO and must be coordinated with other events.

The need for prolonged external fixation and its associated inconvenience, along with the frequent complications can make application of the distraction osteogenesis method onerous. Among the most difficult and frustrating of challenges is delayed or failed bone healing following distraction which can require additional surgeries or ultiinately lead to failure. Strategies to improve the quality, quantity, and rate of formation of bone regenerate are therefore highly desirable.

Induction of neovascularization is recognized as a critical feature of distraction histiogenesis. The mechanism by which distraction induces neovascularization currently is not well understood. HIF-1α is a master regulator of the hypoxic response, and a potent inducer of vasoactive endothelial growth factor (VEGF), leading to vascularization. HIF-1α has been demonstrated to be induced in the distraction gap following acute episodes of distraction in a murine model (Carvalho et al., 2004, Bone 34:849-61) and is shown to have increased expression during fracture healing in a rat model (Komatsu et al., 2004, Bone 34:680-8). Recent studies have shown that small molecules blocking prolyl hydroroxylases (PHDs) involved in HIF-1α degradation can improve healing and prevent damage from ischemia by activating HIF-1α. This approach has been used in skin, heart, brain, and kidneys in animal models in vivo, as well as in phase I and II clinical trials, but it has not been used previously in skeletal repair (reviewed in Giaccia et al., 2003, Nat. Rev. Drug Discov. 2:803-11; Hewitson et al., 2004, Curr. Pharm. Des. 10:821-33).

The hypoxia-inducible factors (HIFs) have been identified as a central pathway for transmitting cellular response to hypoxia and initiating angiogenesis. HIFs are transcription factors which activate genes encoding proteins that mediate adaptive responses to reduced oxygen availability (e.g., angiogenesis). The HIF complex consists of a heterodimer comprised of one of three a subunits (HIF-1α, HIF-2α, or HIF-3α) bound to the aryl hydrocarbon receptor nuclear translocator (ARNT), which is also known as HIF-1β (See FIG. 7). Hypoxia induces changes in the accumulation and activity of the HIF-1α subunit. In most cells, the primary level of regulation is inhibition of the HIF-1α subunit degradation during hypoxia. Generally, the level of HIF-1α protein is low due to the on-going ubiquitination and proteosomal degradation. The molecular mechanisms of ubiquitination and proteosomal degradation of HIF-1α subunits involve enzymatic prolyl hydroxylation on an oxygen degradation domain (ODD) which targets HIF-1α for ligation-mediated proteosomal degradation. The ODD contains a conserved proline residue that is hydroxylated under normoxic conditions by one of a group of three proline hydroxylases (PHDs 1-3). The reaction requires the presence of iron, oxygen, and 2-oxoglutarate. When hydroxylated, the proline residue is recognized by pVHL ubiquitin-protein ligase. During hypoxia, prolyl hydroxylation is blocked, leading to HIF-1α stabilization. A negative feedback loop is established whereby hypoxia and HIF-1α both upregulate PHD2 expression. This results in accumulation of functional HIF-1α subunits, which dimerize with ARNT and translocate to the nucleus. In the nucleus, the HIF-1α/ARNT complex can couple with the coactivator p300 and transactivate HIF-sensitive genes by binding to hypoxia response elements (HREs) in the proximal promoter regions. Hydroxylation of an asparagine residue by the factor inhibiting HIF (FIH) found within the nucleus can block the HIF/p300 association. The elucidation of this pathway has enabled strategies for experimental activation or inhibition of HIF-1α.

In addition to hypoxia per se, mechanical stress and pro-inflammatory cytokines are also known to regulate HIF-1α expression. For example, mechanical stretch of rat myocardium induces HIF-1α accumulation in myocytes through activation of the PI3/AKT/Frap pathway. Similarly, HIF-1α is upregulated in smooth muscle following experimental distension of rat aorta, and in vascular smooth muscle cells subjected to cyclical stretch. Cytokines, such as IL-1β and TNF and/or IFN-γ, have been shown to increase HIF-1α DNA-binding activity in human hepatoma cells and human renal tubular epithelial cells. Also, the pro-inflammatory mediator, nitric oxide (NO), can promote HIF-1α activation tinder normoxic conditions. Both mechanical and pro-inflammatory signals are generated in bone following tissue injury and therefore may contribute to the acute induction of HIF-1α in mouse bone following distraction.

Angiogenesis is recognized as a critical feature of bone healing and occurs in close spatial and temporal association with osteogenesis during distraction. However, the mechanisms that regulate the coupling of angiogenesis to osteogenesis are not well understood. Recent studies implicate HIF-1α as a central player in neovascularization in DO. Pacicca et al. used immunohistochemistry to identify two known angiogenic factors, bFGF (basic Fibroblast Growth Factor) and VEGF, in a rat femur distraction model (2003, Bone 33:889-98). They noted bFGF in cells adjacent to the new longitudinal vessels and VEGF in the osteoblasts and undifferentiated cells in the distractrion gap. Microarray analysis revealed doubling or tripling of expression of angiogenesis-associated mRNAs including angiopoietin 1, pleiotrophin, tie-1, and tie-2 during distraction as compared to latency. Also noted was a very high level of expression of HIF-1α.

In a related study in a mouse model, immediate response following a cycle of distraction was examined. While a large number of genes were induced after the first cycle of distraction, only HIF1-α, VEGF-A, ang 1, and neuropilin were found to be induced even after the last cycle (Carvalho et al., 2004, Bone 34:849-61). Furthermore, HIF1-α expression was found to be upregulated, and HIF1-α immunostaining increased in cells lining the areas of bone formation in the distraction gap following a mechanical intervention (axial shortening following distraction) that resulted in improved healing in a rabbit DO model.

Prior studies have attempted to manipulate the anglogenic response to improve bone healing using VEGF, a downstream target of HIF1-α with both positive and negative results. Healing of fractures and critical sized defects has been improved by local application and genetic therapy (Mori et al., 2006, J. Orthop. Res. 24:653-63; Eckardt et al., 2005, J. Bone Joint Surg. Br. 87:1434-38). Use in a routine distraction protocol in the rabbit tibia did not improve healing (Street et al., 2002, Proc. Natl. Acad. Sci. USA 99:9656-61). The lack of effect in routine distraction may be because the model predictably heals well, and VEGF is already being stimulated and may represent a type II error. Additionally, VEGF alone may be insufficient to induce and sustain angiogenesis because other factors such as Angiopoetins and Tie 1 and 2 are required to restabilize endothelial cells following recruitment (Yancopoulos et al., 2000, Nature 407:242-48).

Pharmacologic strategies have been devised to manipulate the HIF-1α pathway to either promote or retard angiogenesis (See, e.g., Giaccia et al., 2003, Nat. Rev. Drug, Discov. 2:803-11). In general, therapies to increase HIF-1α activation include gene therapy, custom peptides, or small molecules. A constitutively active HIF-1α gene delivered with an adenovirus vector was effective in rabbit limb ischemia (Vincent et al., 2000, Circulation 102:2255-61) and is currently in phase II clinical trials for intermittent claudication (ClinicalTrials.gov identifier NCT00117650). Potential disadvantages of these approaches include the risk (real or perceived) associated with viral vectors, cost, and the need for a carrier to contain the material to the distraction site (typically applied by a sponge or intramuscular injection). Another approach has been the development of small custom peptides that mimic the VHL-binding prolyl hydroxylation sites in HIF-1α (the ODD). The peptides saturate the HIF-PHDs, blocking degradation of HIF-1α and inducing angiogenesis in a mouse implanted sponge model (Warnecke et al., 2003, Faseb J. 17:1186-88; Willam et al., 2002, Proc. Natl. Acad. Sci. USA 99:10423-28).

Two prototypical agents already in clinical use, desferrioxamine and cobalt, have been found to mimic hypoxia and increase HIF-1α. Both are first generation hypoxic mimetics that exert their effects by interfering with iron requiring enzymes, including the PHDs. They have been shown to increase HIF-1α activation in vitro and in vivo (See, e.g., Ivan et al., 2001, Science 292:464-68; Maxwell et al., 1999, Nature 399:271-75; Hirsila et al., 2005, Faseb J. 19:1308-10) and have exhibited positive effects on angiogenesis and response to ischemia (See, e.g., Matsumoto et al., 2003, J. Arm. Soc. Nephrol. 14:1825-32; Roscnberger et al., 2002, J. Am. Soc. Nephrol. 13:1721-32). Clinical uses have included treatment of anemia (Duckham et al., 1976, Q. J. Med. 45:277-94), iron overload, and a small trial of local treatment for limb ischemia (Klpshidze et al., 2003, Int. Angiol. 22:349-55). A disadvantage to these candidate agents is that iron chelation has been shown to have some toxicity In children in lung term applications for anemia (Bentur et al., 1991, Drug Saf. 6:37-46; Olivieri et al., 1986, N. Engl. J. Med. 314:869-73).

Another class of agents are 2-oxoglutarate analogs which were developed to decrease scar formation by targeting C4PHD. These agents have been shown in vitro to vary in their inhibition of C4PHD and the PHDs (Hirsila et al., 2003, J. Biol. Chem. 278:30772-80). In vivo, they have increased HIF-1α activation, been protective against ischemia, and demonstrated no toxicity. Additional agents that impinge on the HIF-1α pathway are being discovered and developed (e.g., L-mimosine, 3,4 dihydroxybenzoate (3,4 DHB), and dimethyloxalylglycine (DMOG)). Using high throughput screening strategies where cell lines were stably infected with a HRE-luciferase construct, Melillo et al. found that the NCI diversity set and small molecule library contained many agents that inhibited HRE activation (Kong et al., 2005, Cancer Res. 65:9047-55; Rapisarda et al., 2002, Cancer Res. 62:4316-24). Furthermore, there is evidence that relative abundance of the three PHDs and their responsiveness to hypoxia is tissue specific (Appelhoff et al., 2004, J Biol. Chem. 279:38458-65). PHD2 seems to predominate in many cell culture lines, but PHDs 1 and 3 may in fact be more oxygen sensitive.

What is needed in the art, therefore, are methods for identifying pharmacologic agents that increase bone healing, increase osteoblast cell differentiation, improve bone mass or volume, and/or promote osteogenesis. Also needed are the compounds or small molecules identified by such methods and the use of such compounds or small molecules in methods for improving bone healing.

### SUMMARY OF THE INVENTION

The present invention provides for methods of identifying a compound that increases bone healing, increases osteoblast cell differentiation, improves bone mass or volume, and/or promotes osteogenesis. Such methods comprise providing an osteoblast or bone precursor cell, contacting the cell with a test compound, and determining whether an increase in dimerization, nuclear translocation, and/or transcriptional activity of a HIF-1α/HIF-1β complex occurs in the cell contacted with the compound, said increase being an indication that the compound increases bone healing, increases osteoblast cell differentiation, improves bone mass or volume, and/or promotes osteogenesis. It is contemplated that promoting osteogenesis can comprise one or more of increasing osteoblast formation, increasing osteoid volume, decreasing ostoclast formation, and decreasing osteoclast function. Transcriptional activity of the HIF-1α/HIF-1β complex can be determined by expression of a HIF-1α target gene selected from the group consisting of VEGF; nitric oxide synthase 2; heme oxygenase 2; α_{1B}-adrenergic receptor; erythropoietin; transferrin; ceruloplasmin; transferrin receptor; cyclin G2; p21; IGF-2; IGF-binding protein 1, 2 and 3; glucose transporter 1, and 3; hexokinase 1, and 2; phosphofructokinase L; pyruvate kinase M; a HIF-prolyl hydroxylase; and carbonic anhydrase. In certain embodiments, the transcriptional activity is determined by measuring expression of VEGF or Glut-1.

The present invention also provides for methods of identifying a compound that decreases osteoclast cell differentiation, decreases bone mass or volume, and/or increasing osteoclast cell differentiation. Such methods comprise providing an osteoblasts or bone precursor cell, contacting the cell with a test compound, and determining whether a decrease in dimerization, nuclear translocation, and/or transcriptional activity of a HIF-1α/HIF-1β complex occurs in the cell contacted with the compound, said decrease being an indication that the compound decreases osteoblast cell differentiation, decreases bone mass or volume, and/or increases osteoclast cell differentiation.

In another embodiment, the invention provides a method of identifying a compound that modulates the dimerization of HIF-1α, the nuclear translocation of HIF-1α or the transcriptional activity of HIF-1α in bone is identified. In certain embodiments, the compound increases dimerization, nuclear translocation, and or transcriptional activity of HIF-1α. In other embodiments, the compound decreases dimerization, nuclear translocation, and or transcriptional activity of HIF-1α.

The invention further contemplates a process for making a compound that increases osteoblast coli differentiation, improves bone mass or volume, and/or promotes osteogenesis, comprising carrying out any of the methods described above to identify a compound that increases osteoblast cell differentiation, improves bone volume, and/or promotes osteogenesis, and manufacturing the compound. The invention additionally contemplates a process for making a compound that decreases osteoblast cell differentiation, decreases bone mass or volume, and/or increases osteoclast cell differentiation, comprising carrying out any of the methods described above to identify a compound that decreases osteoblast cell differentiation, decreases bone volume, and/or increases osteoclast cell differentiation, and manufacturing the compound. Pharmaceutical compositions comprising said compounds, and methods of administering said pharmaceutical compositions to an individual in need thereof are also contemplated.

The compositions and methods described herein can also serve for therapeutic intervention in bone-related disorders. For example, compounds that promote the activity of HIF-1α can be Formulated into a pharmaceutical formulation for the treatment of a disease state, such as, but not limited to osteoporosis, osteopenia, or other bone-related disorders.

Another aspect of this invention is directed to methods for strengthening a bone graft, inducing vertebral synostosis, enhancing long bone extension, the treatment and promotion of healing of bone fractures and osteotomies, enhancing bone healing following facial reconstruction, maxillary reconstruction and/or mandibular reconstruction in a vertebrate, e.g., a mammal (including a human being), comprising administering to said vertebrate a therapeutically effective amount of a compound of the current invention that promotes the activity of HIF-1α, a prodrug or a pharmaceutically acceptable salt thereof, or a stereoisomer or diastereomeric mixture of said compound, prodrug or salt. The composition may be applied locally to the site of bone reconstruction or may be administered systemically.

In a further example, compounds that decrease the activity of HIF-1α can be formulated into a pharmaceutical formulation for the treatment of a disease state characterized by bone overgrowth, such as, but not limited to sclerotic bone.

### BRIEF DESCRIPTION OF THE DRAWINGS

FIG. 1 shows that conditional deletion of VHL in osteoblasts increases bone volume and vascularity. Panel A is a schematic that depicts the constructs used to create a conditional deletion of VHL in osteoblasts. The human osteocalcin (hOC) promoter was used to generate mice carrying a Cre transgene (top). LoxP restriction sites were inserted flanking the first exon of VHL. Osteocalcin Cre mediated recombination results in loss of VHL function is osteoblasts (bottom). Panel B is a photograph of the bones of the hindlimb from a control (left) and a ΔVHL mouse (right). The bones of the ΔVHL mouse are noted on gross inspection to be thicker and appear blood-filled when compared with bones from the control littermate. Panel C shows the results of MicroCT imaging of femurs from ΔVHL mice (right) compared to control littermates (left). These images show significantly increased bone volume in ΔVHL mice compared to control littermates at 6 weeks of age as noted on longitudinal and cross sectional images. Panel D shows the results of MicroCT angiography of hindlimbs of ΔVHL mice (right) and control littermates (left). The MicroCT imaging was performed following Microfil perfusion. Reconstruction of vasculature within the bone from concordant areas of the distal femur shows increased vascularity in the ΔVHL mouse.

FIG. 2 shows that the Cre mediated deletion of VHL in osteoblasts in vitro activates HIF-1α. Panel A shows a photograph of Western blots stained with antibodies directed to either HIF-1α or α-tubulin. Primary osteoblasts from VHLF1/F1 mice were treated with either adeno GFP or adeno Cre to delete VHL. Western blotting of cytoplasmic and nuclear protein for HIF-1α compared to control α-tubulin confirmed HIF-1α activation. Panel B is a graph showing an increase in HIF-1α-activated genes. To evaluate gene expression, mRNA was collected 48 hours after transfection, and real-time PCR was performed, confirming the VHL deletion, and demonstrating upregulation of VEGF and Glut-1. Expression of HIF-1α and HIF2α changed modestly. Panel C is a photograph of cells stained for alkaline phosphatase activity or mineralization. To evaluate the impact of the VHL deletion on osteogenic differentiation following transfection, cells were cultured in the presence of β-glycerol phosphate and ascorbate for 14 days. Differentiation was not significantly different by staining for alkaline phosphatase (ALP) activity or mineralization (Von Kossa).

FIG. 3 is a photograph showing that cells in the distraction gap are hypoxic, Pimonidazole was administered to mice immediately before distraction. Three hours later, the mice were sacrificed. Immunostaining for pimonidazole-protein adducts was performed on sections from the distraction area. Panel A shows that no staining was seen in controls (redundant primary antibody). Panel B shows that intense DAB staining was seen in cells in the distraction gap. Panel C shows that at higher magnification, the cells lining the surface of newly formed bone were positively stained (arrows).

FIG. 4 shows that activation of HIF-1α increases angiogenesis and improves bone healing in distraction osteogenesis. Panel A shows X-rays and MicroCT images depicting healing of a distraction gap in control (top) and ΔVHL mice. Distraction osteogenesis was performed at a rate of 0.15 mm/day. X-rays demonstrate healing of the distraction gap by plain radiographs at the end of consolidation (left). MicroCT images of bone healing in the distraction gap are not markedly different at the conclusion of consolidation (middle). However, microCT angiography performed using a silicone perfusion technique shows increased vascularity in the distraction zone at the end of distraction in the ΔVHL mice (right), Panel B shows a quantitation of the MicroCT data. Bone volume per total volume in the distraction gap at the end of consolidation was not different in the ΔVHL mice compared to controls (top), but vessel volume per total volume in the distraction zone at the end of distraction was dramatically increased (bottom). Panel C shows X-rays and MicroCT images at the conclusion of the consolidation period. The distraction rate was increased to 0.3 mm/day. Radiographs (left) and microCT images (right) at the conclusion of the consolidation period show increased bone formation in the distraction zone in the ΔVHL mice following distraction.

FIG. 5 shows that prolyl hydroxylases are present in osteoblasts in vitro. Panel A is a photograph of Western blots stained for PHD1, PHD2, or α-tubulin. Western blotting for PHDs 1 and 3 was performed using protein from cultured osteoblasts with and without VHL deletion. Panel B is a schematic showing the active site for hydroxylation by the PHDs, represented by the grey area, with note of the cofactors iron, oxygen, and 2-oxyglutarate. Panel C is a schematic showing the structures of 2-oxyglutarate and analogs 3,4-DHB and L-mimosine (from Warnecke et al., 2003, Faseb J. 17:1186-8).

FIG. 6 shows that cobalt activates HIF-1α in osteoblasts in vitro. Primary osteoblast cultures wore exposed to 5, 10, or 50 ng/ml of cobalt. Panel A shows a Western blot of nuclear extracts stained for HIF-1α, showing increased nuclear accumulation after cobalt treatment at all doses. Panel B is a graph of real time PCR data. Real time PCR was performed for VEGF following cobalt treatment. Marked upregulation of VEGF was seen after 12 and 24 hours of treatment.

FIG. 7 is a schematic showing the regulation of HIFs by molecular oxygen.

FIG. 8 shows that vessel volume and bone volume are increased in ΔVHL mice. Panel A is an autoradiograph of bones from ΔVHL mice and control mice (Con). Panel B shows the results of microCT angiography; and Panel C shows microCT volumetric and sagital plane reconstruction images. Panel D shows graphs of vessel volume per total volume (VV/TV) and bone volume per total volume (BV/TV). Both show significant increases associated with the targeted deletion of VHL.

### DETAILED DESCRIPTION

The present invention may be understood more readily by reference to the following detailed description of the preferred embodiments of the invention and the Examples included herein. However, before the present compositions and methods are disclosed and described, it is to be understood that this invention is not limited to specific cell types, specific host cells, specific nucleic acids, specific polypeptides, specific conditions, or specific methods, etc., as such may, of course, vary, and the numerous modifications and variations therein will be apparent to those skilled in the art.

The present invention relates to a method for screening for a compound that increases bone healing, increases osteoblast differentiation, improves bone mass or volume, and/or promotes osteogenesis.

The present invention provides methods for identifying a compound that improves bone mass or volume, wherein the method comprises the steps of: providing an osteoblast or bone precursor cell; contacting the cell with a test compound; and determining whether an increase in dimerization, nuclear translocation, and/or transcriptional activity of a HIF-1α/HIF-1β complex occurs in the cell contacted with the compound, said increase being an indication that the compound improves bone mass or volume. In certain embodiments, the transcriptional activity of a HIF-1α/HIF-1β complex is determined by measuring the transcription activation of a HIF-1α target gene selected from the group consisting of VEGF; nitric oxide synthase 2; heme oxygenase 2; a1B-adrenergic receptor; erythropoietin; transferrin; ceruloplasmin; transferrin receptor; cyclin G2; p21; IGF-2; IGF-binding protein 1, 2 and 3; glucose transporter 1, and 3; hexokinase 1, and 2; phosphofructokinase L; pyruvate kinase M; a HIF-prolyl hydroxylase; and carbonic anhydrase. The transcription activation may be determined by techniques well known in the art, for example, by real-time PCR, Northern blotting, or reporter gene assays. In other embodiments, the expression level of HIF-1α protein is measured. In certain embodiments, the expression level is measured by Western blotting.

Because it is possible that compounds identified by the present methods may also affect collagen synthesis, the present invention also provides methods that comprise an additional step of determining whether an increase or decrease in collagen synthesis occurs in the cell contacted with the compound. In certain embodiments, the increase or decrease in collagen synthesis is detected by measuring the level of secreted collagen in the media, for example, by Western blotting.

The present invention also provides processes for making a compound that increases bone healing, increases osteoblast cell differentiation, improves bone mass or volume, and/or promotes osteogenesis, comprising the steps of: providing an osteoblast or bone precursor cell; contacting the cell with a test compound; determining whether an increase in dimerization, nuclear translocation, and/or transcriptional activity of a HIF-1α/HIF-1β complex occurs in the cell contacted with the compound, said increase being an indication that the compound increases bone healing, increases osteoblast cell differentiation, improves bone mass or volume, and/or promotes osteogenesis; and manufacturing the compound.

The present invention provides methods of identifying a compound that promotes osteogenesis, wherein the method comprises the comprises the steps of: providing an osteoblast or bone precursor cell; contacting the cell with a test compound; and determining whether an increase in dimerization, nuclear translocation, and/or transcriptional activity of a HIF-1α/HIF-1β complex occurs in the cell contacted with the compound, said increase being an indication that the compound promotes osteogenesis. The compounds of the present invention promote osteogenesis by one or more of increasing osteoblast formation, increasing osteoid volume, decreasing osteoclast formation, and decreasing osteoclast function.

The present invention also provides methods of identifying a compound that increases osteoblast cell differentiation, wherein the method comprises the steps of: providing an osteoblasts or bone precursor cell; contacting the cell with a test compound; and determining whether an increase in dimerization, nuclear translocation, and/or transcriptional activity of an HIF-1α/HIF-1β complex occurs in the cell contacted with the compound, said increase being an indication that the compound increases osteoblast cell differentiation. The present invention also provides for methods of identifying a compound capable of decreasing osteoblast cell differentiation, decreasing bone mass or volume, and/or increasing osteoclast cell differentiation, wherein the methods comprise the steps of providing an osteoblast or bone precursor cell; contacting the cell with a test compound; and determining whether a decrease in dimerization, nuclear translocation, and/or transcriptional activity of a HIF-1α/HIF-1β complex occurs in the cell contacted with the compound, said decrease being an indication that the compound decreases osteoblast cell differentiation, decreases bone mass or volume, and/or increases osteoclast cell differentiation.

The present invention provides processes for making a compound that decreases osteoblast cell differentiation, decreases bone mass or volume, and/or increases osteoclast cell differentiation, comprising the steps of: providing an osteoblast or bone precursor cell; contacting the cell with a test compound; and determining whether a decrease in dimerization, nuclear translocation, and/or transcriptional activity of a HIF-1α/HIF-1β complex occurs in the cell contacted with the compound, said decrease being an indication that the compound decreases osteoblast cell differentiation, decreases bone mass or volume, and/or increases osteoclast cell differentiation; and manufacturing the compound.

The present invention provides compounds for use in promoting the activity of HIF-1α for the treatment of a disease state, such as, but not limited to osteoporosis, osteopenia, or a bone-related disorder selected from the group consisting of osteoporosis, bone fractures, hypercalcemia of malignancy, osteopenia or osteolytic lesions due to bone metastases, periprosthetic osteolysis, familial expansile osteolysis, periodontal disease, tooth loss, rheumatoid arthritis, osteoarthritis, hyperparathyroidism, Paget's disease, osteodystrophy, myositis ossificans, Bechterew's disease, malignant hypercalcernia, bone loss, bone abnormalities due to steroid hormone treatment, bone abnormalities caused by cancer therapeutics, abnormally increased bone turnover, osteomalacia, Bechet's disease, hyperostosis, osteopetrosis, osteogenesis imperfecta, rachitis, immobilization-induced osteopenia, expansile skeletal hyperphosphatasia, and glucocorticoid-induced osteoporosis.

The present also provides methods for strengthening a bone graft, inducing vertebral synostosis, enhancing long bone extension, the treatment and promotion of healing of bone fractures and osteotomles, enhancing bone healing following facial reconstruction, maxillary reconstruction, and/or mandibular reconstruction in a vertebrate, comprising the step of: administering to said vertebrate a composition comprising a therapeutically effective amount of a compound of the present invention, a prodrug, or a pharmaceutically acceptable salt thereof, or a stereoisomer or diastereomeric mixture of said compound, prodrug, or salt. In certain embodiments, the composition is administered locally to the site of bone reconstruction. In other embodiments, the composition is administered systemically.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part 1; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering Principles and Methods, Vols. 14, Plenum Press, New York. Abbreviations and nomenclature, where employed, are deemed standard in the field and commonly used in professional journals such as those cited herein.

Osteoblasts originate from mesenchymal progenitors or osteoprogenitor cells that, with the appropriate stimulation, undergo proliferation and differentiate into preosteoblasts, and then into mature, functional osteoblasts. In culture, as in vivo, osteoblasts form bone-like mineralized nodules by undergoing three stages of development; proliferation, extracellular matrix maturation, and mineralization. During each stage of development, specific subsets of genes are sequentially expressed or repressed. For example, collagen I is known to be a marker for proliferation, alkaline phosphatase for extracellular matrix maturation, and osteocalcin for mineralization. The regulation of gene expression in osteoblasts during development and differentiation occurs predominantly at the transcriptional level. Several transcription factors and signaling pathways, such as HIF-1α, AP-1, Runx2, and β-catenin have been shown to play a major role in the regulation of osteoblast gene expression, phenotype, and ultimately bone formation.

As used herein, the term “osteoblast” or “osteoblast cell” refers to a terminally or non-terminally differentiated cell derived from a bone precursor cell, wherein the osteoblast cell is at least more differentiated towards an osteoblast phenotype than the cell from which it is derived. As used herein, “osteoblasts” or “osteoblast cells” are characterized by the expression of one or more specific marker transcripts, such as, but not limited to, AP-1 family members, Runx2, Fra-2, alkaline phosphatase, osteocalcin, β-catenin, CCAAT/enhancer binding protein (C/EBP), and ATF4, and may also show matrix deposition, matrix mineralization, and/or cuboidal morphology of the cells. Furthermore, as used herein, the term “terminally differentiated osteoblast” refers to an osteoblast cell that is actively producing and mineralizing bone material.

Also, as used herein, producing an osteoblast cell encompasses the production of a cell culture that is enriched for osteoblast cells. In certain embodiments of the present invention, the term “enriched” refers to a cell culture that contains more than approximately 50%, 55%, 60%, 65%, 70%, 75%, 80%, 85%, 90%, or 95% of the desired cell lineage.

As used herein, the term “bone precursor cell” refers to a cell that differentiates towards the osteoblast lineage upon treatment with known osteoblast-promoting agents, such as, but not limited to type I collagen, fibrinogen, fibrin, fibrinogen, osteocalcin, osteonectin, TGF-β, 1,25-OH Vitamin D3, basic fibroblast growth factor, or bone morphogenic protein 2. It is preferred that the bone precursor cell express one or more of osteocalcin, osteonectin, or alkaline phosphatase. In a preferred embodiment, bone precursor cells include osteoprogenitor cells or preosteoblasts. As used herein, the term “differentiate” refers to the production of a cell type that is more differentiated than the cell type from which it is derived. The term therefore encompasses cell types that are partially and terminally differentiated.

As used herein, the terms “compound” or “pharmacologic agent” refer to any compound or molecule that may affect bone mass or volume, osteogenesis, osteoblast differentiation, bone graft strengthening, vertebral synostosis, long bone extension, the treatment and promotion of healing of bone fractures and osteotomies, or bone healing following facial reconstruction, maxillary reconstruction, and/or mandibular reconstruction.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCR, in situ hybridization, real time PCR, Western blotting, and immunostaining.

As used herein, the term “contacting” (i.e., contacting a cell e.g. a target cell, with a compound) is intended to include incubating the compound and the cell together in vitro (e.g., adding the compound to cells in culture). The term “contacting” is not intended to include the in vivo exposure of cells to an effector of the HIF-1α signaling pathway that may occur naturally in a subject (i.e., exposure that may occur as a result of a natural physiological process). The step of contacting the cell with a test compound can be conducted in any suitable manner. For example, the cells may be treated in adherent culture or in suspension culture.

A cell differentiating medium or environment may be utilized to partially, terminally, or reversibly differentiate the bone progenitor cells of the present invention. In accordance with the invention the medium of the cell differentiation environment may contain a variety of components including, for example, DMEM, Ham's F12 medium, FBS (fetal bovine serum), FGF2 (fibroblast growth factor), α-MEM, vitamin C, beta glycerophosphate. The cell differentiation environment can also contain supplements such as L-Glut-amine, NEAA (non-essential amino acids), and P/S (penicillin/streptomycin). It is contemplated that additional factors may be added to the cell differentiation environment, including, but not limited to, fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family.

In other embodiments, the cell differentiation environment comprises plating the cells in an adherent culture. As used herein, the terms “plated” and “plating” refer to any process that allows a cell to be grown in adherent culture. As used herein, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates. The substrate for the adherent culture may comprise any one or combination of polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, or laid down by the target cell or cell culture.

The methods of the present invention contemplate that target cells may be cultured with a feeder cell or feeder layer. As used herein, a “feeder cell” is a cell that is co-cultured with a target cell and stabilizes the target cell in its current state of differentiation. A feeder layer comprises more than one feeder cell in culture. In one embodiment of the above method, conditioned medium is obtained from a feeder cell that stabilizes the target cell in its current state of differentiation. Any and all factors produced by a feeder cell that allow a target cell to be stabilized in its current state of differentiation can be isolated and characterized using methods routine to those of skill in the art. These factors may be used in lieu of a feeder layer, or may be used to supplement a feeder layer.

With respect to some of the embodiments of differentiation methods described herein, the above-mentioned growth factors are provided to the cells so that the growth factors are present in the cultures at concentrations sufficient to promote differentiation of at least a portion of the target cells to the desired cell lineage. In some embodiments of the present invention, the above-mentioned growth factors are present in the cell culture at a concentration of at least about 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml. In certain embodiments of the present invention, the above-mentioned growth factors are removed from the cell culture subsequent to their addition. For example, the growth factors can be removed within about one day, about two days, about three days, about four days, about five days, about six days, about seven days, about eight days, about nine days or about ten days after their addition.

The compositions and methods described herein have several useful features. For example, the compositions and methods described herein are useful for identifying compounds for the therapeutic intervention in disease states, such as osteoporosis, osteopenia, or other bone-loss or bone density decreasing disorders. As used herein, the phrase “bone-related disorder” refers to a disorder wherein bone formation, deposition, or resorption is abnormal. Bone-related disorders include, but are not limited to, osteoporosis, bone fractures, hypercalcemia of malignancy, osteopenia or osteolytic lesions due to bone metastases, periprosthetic osteolysis, familial expansile osteolysis, periodontal disease, tooth loss, rheumatoid arthritis, osteoarthritis, hyperparathyroidism, Paget's disease, osteodystrophy, myositis ossificans, Bechterew's disease, malignant hypercalcemia, bone loss, bone abnormalities due to steroid hormone treatment, bone abnormalities caused by cancer therapeutics, abnormally increased bone turnover, osteomalacia, Bechet's disease, hyperostosis, osteopetrosis, osteogenesis imperfecta, rachitis, immobilization-induced osteopenia, expansile skeletal hyperphosphatasia, and glucocorticoid-induced osteoporosis.

In the methods of the present invention, the compounds described herein and determined using the screening methods described herein, can form the active ingredient, and are typically administered in admixture with suitable pharmaceutically acceptable diluents, excipients, adjuvants, or carriers (collectively referred to herein as “carrier” materials) suitably selected with respect to the intended form of administration, that is, oral tablets, capsules, pills, powders, granules, elixirs, tinctures, suspensions, syrups and the like, and consistent with conventional pharmaceutical practices. Likewise, they may also be administered in intravenous (bolus or infusion), intraperitoneal, intranasal, rectal, topical, subcutaneous, intramuscular or transdermal form, all using forms well known to those of ordinary skill in the pharmaceutical arts. In a preferred embodiment, the compounds are administered locally to the site of bone reconstruction. In another preferred embodiment, the compounds are administered systemically.

For oral administration in the Corm of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like. When aqueous suspensions are required for oral use, the active ingredient is combined with emulsifying and suspending agents. If desired, certain sweetening and/or flavoring agents may be added. For intramuscular, intraperitoneal, subcutaneous, and intravenous use, sterile solutions of the active ingredient are usually prepared, and the pH of the solutions should be suitably adjusted and buffered. For intravenous use, the total concentration of solutes should be controlled in order to render the preparation isotonic.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

The compounds can be applied to the sites of bone fractures or osteotomies, for example, either by injection of the compound in a suitable solvent (e.g., an oily solvent such as arachis oil) to the cartilage growth plate or, in cases of open surgery, by local application thereto of the compound in a suitable vehicle, carrier or diluent such as bone-wax, demineralized bone powder, polymeric bone cements, bone sealants, etc. Alternatively, local application can be achieved by applying a solution or dispersion of the compound in a suitable carrier or diluent onto the surface of, or incorporating it into solid or semi-solid implants conventionally used in orthopedic surgery, such as dacron-mesh, gel-foam and kie] bone, or prostheses.

As used herein, the phrase “pharmaceutically acceptable” refers to an agent that does not interfere with the effectiveness of the biological activity of an active ingredient, and which may be approved by a regulatory agency of the Federal government or a state government, or is listed in the U.S. Pharmacopeia or other generally recognized pharmacopeia for use in animals, and more particularly for use in humans. Accordingly, suitable pharmaceutically acceptable carriers include agents that do not interfere with the effectiveness of a pharmaceutical composition.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines.

Compounds of the present invention also may be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and crosslinked or amphipathic block copolymers of hydrogels.

Methods of preparing various pharmaceutical compositions with a certain amount of active ingredient are known to those skilled in the art. For examples of methods of preparing pharmaceutical compositions, see Remington: The Science and Practice of Pharmacy, Mack Publishing Company, Easton, Pa., 19th Edition (1995).

The instant compounds are also useful in combination with known agents useful for treating bone-related disorders. Combinations of the presently disclosed compounds with other agents useful in treating osteoporosis or other bone disorders are within the scope of the invention. A person or ordinary skill in the art would be able to discern which combinations of agents would be useful based on the particular characteristics of the drugs and the disease involved. Such agents include but are not limited to the following: an organic bisphosphonate; a cathepsin K inhibitor; an estrogen or an estrogen receptor modulator; an androgen receptor modulator; an inhibitor of osteoclast proton ATPase; an inhibitor of HMG-CoA reductase; an integrin receptor antagonist; an osteoblast anabolic agent, such as PTH; calcitonin; Vitamin D or a synthetic Vitamin D analogue; selective serotonin reuptake inhibitors (SSRIs); and the pharmaceutically acceptable salts and mixtures thereof.

The term “administration” and variants thereof (e.g., “administering” a compound) in reference to a compound of the invention means introducing the compound or a prodrug of the compound into the system of the individual in need of treatment. When a compound of the invention or prodrug thereof is provided in combination with one or more other active agents (e.g., a bisphosphonate, etc.), “administration” and its variants are each understood to include concurrent and sequential introduction of the compound or prodrug thereof and other agents.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

When a compound according to this invention is administered into a human subject, the daily dosage will normally be determined by the prescribing physician with the dosage generally varying according to the age, sex, weight, and response of the individual patient, as well as the severity of the patient's symptoms, the route of administration; and the particular compound or salt thereof employed. An ordinarily skilled physician, veterinarian or clinician can readily determine and prescribe the effective amount of the drug required to prevent, counter, or arrest the progress of the condition.

In one exemplary application, a suitable amount of compound is administered to a mammal undergoing treatment. Oral dosages of the present invention, when used for the indicated effects, will range between about 0.01 mg per kg of body weight per day (mg/kg/day) to about 100 mg/kg/day, preferably 0.01 to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three, or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

The compositions and methods of the present invention are administered and carried out until the desired therapeutic effect is achieved. The phrase “until the desired therapeutic effect is achieved,” as used herein, means that the therapeutic agent or agents are continuously administered, according to the dosing schedule chosen, up to the time that the clinical or medical effect sought for the disease or condition being treated is observed by the clinician or researcher. For methods of treatment of the present invention, the pharmaceutical composition is continuously administered until the desired improvement in bone mass or structure is observed. In such instances, achieving an improvement in bone mass or a replacement of abnormal bone structure with normal bone structure are the desired objectives. For methods of prevention of the present invention, the pharmaceutical composition is continuously administered for as long as necessary to prevent the undesired condition. In such instances, maintenance of bone mass density is often the objective. Non-limiting examples of administration periods can range from about 2 weeks to the remaining lifespan of the mammal. For humans, administration periods can range from about 2 weeks to the remaining lifespan of the human, preferably from about 2 weeks to about 20 years, more preferably from about 1 month to about 20 years, more preferably from about 6 months to about 10 years, and most preferably from about 1 year to about 10 years.

As used herein, the term “composition” is intended to encompass a product comprising the specified ingredients in the specified amounts, as well as any product which results, directly or indirectly, from combination of the specified ingredients in the specified amounts. The term “therapeutically effective amount” as used herein means that amount of active compound or pharmaceutical agent that elicits the biological or medicinal response in a tissue, system, animal or human that is being sought by a researcher, veterinarian, medical doctor or other clinician. The terms “treating” or “treatment” of a disease as used herein includes: preventing the disease, i.e., causing the clinical symptoms of the disease not to develop in a mammal that may be exposed to or predisposed to the disease but does not yet experience or display symptoms of the disease; inhibiting the disease, i.e., arresting or reducing the development of the disease or its clinical symptoms; or relieving the disease, i.e., causing regression of the disease or its clinical symptoms.

As used herein, the term “improving” with respect to bone mass or volume includes increasing or maintaining the current bone mass or volume of an individual, and includes slowing the rate of bone loss. As such, the term includes reducing or inhibiting the resorption of bone in bone-related disorders. As described herein, determining whether an increase in dimerization, nuclear translocation, and/or transcriptional activity of a HIF-1α/HIF-1β complex occurs in the cell contacted in vitro with the compound is predictive that the compound is useful for treating a bone-related disorder, or improving bone mass or volume. The term “bone resorption,” as used herein, refers to the process by which osteoclasts degrade bone. As used herein, the term “bone mass” refers to bone mass per unit area, which is sometimes referred to as bone mineral density.

### Statistical Analysis

T-tests are used to compare the primary outcome of interest, i.e., bone healing response (operationalized as the proportion of Bone Volume/Total Volume in the distraction gap), between the treatment and control groups. Additional outcomes include angiogenesis (VV/TV), histologic healing (percent bone per area of distraction gap), and biomechanical properties (stiffness, hardness, energy to failure). Separate, independent analyses are conducted for each of the treatment groups relative to each control group. There are no specific hypotheses requiring a comparison of the treatment groups with each other, nor a comparison of the control groups. Therefore, the use of independent t-tests is considered more desirable than an analysis of variance (ANOVA). However, post-hoc analyses are conducted using this type of statistical model. It should be noted that these statistical tests place certain assumptions upon the study data, particularly the dependent variable. Prior work in this area has used parametric statistics suggesting that these assumptions have been met in those studies. However, should this not be the case, non-parametric tests equivalent to those described above are used (e.g., Wilxocon test, Kruskal-Wallis test). Finally, appropriate correction of the p-value for statistical significance is employed to account for the multiple statistical comparisons being conducted.

Information regarding expected values for bone healing response may be obtained from prior relevant work in this area (See, e.g., Alkhiary et al., 2005, J. Bone Joint Surg. Am. 87:731-41; Thrailkill et al., 2005, Diabetes 54:2875-81; Isefuku et al., 2004, J. Orthop. Res. 22:1276-82). The table below presents the sample size requirements (per group) based upon a range of possible effect sizes. These sample size estimates are based upon having 80% power to detect these differences at a 5% (two-sided) significance level. The anticipated effect size is approximately 20%, thus 10 animals per group will be required. With this sample size there will be adequate (i.e., 80%) power to detect this effect size and ample power to detect effect sizes that are larger in magnitude. For example, given a sample size of 10 animals per group, the power to detect a 25% difference is 94%; for a 30% difference the power is 99%. For smaller effect sizes (i.e., 10-15%) power is diminished but is still reasonable (e.g., 30-60%). Effect sizes of less than 20% would be of marginal clinical interest, however.

TABLE 1
Effect Size (Percent Difference Between
Treatment and Control Groups)

| | 5% | 10% | 15% | 20% | 25% |
|---|---|---|---|---|---|
| Sample size per | 142 | 36 | 18 | 10 | 7 |
| group | | | | | |

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Development and Characterization of a Mouse Model of Increased HIF-1α in Bone

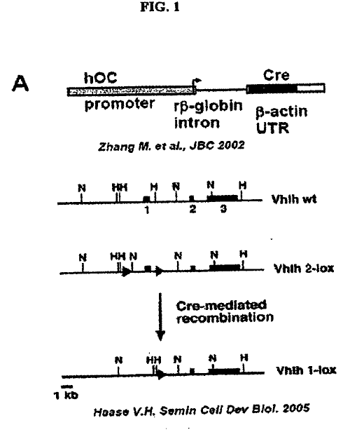

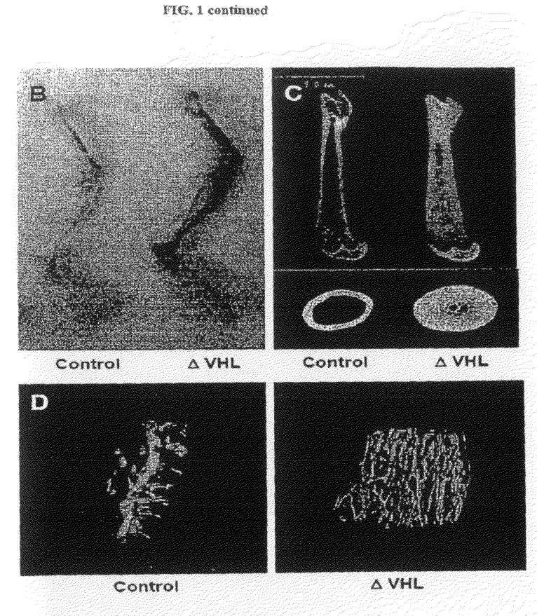

A mouse was developed in which HIF-1α was increased in bone through genetic manipulation. A strategy to impair degradation of HIF-1α, rather than increase its expression was required because degradation is the main mode of regulation of HIF-1α. This impaired degradation was achieved using a targeted Cre/loxP approach to selectively delete VHL (which is responsible for HIF-1 degradation) in osteoblasts (FIG. 1A).

Mice expressing the Cre recombinase in osteoblasts (osteocalcin Cre (OC-Cre)) were crossed with mice homozygous for floxed VHL. The resulting Oc-Cre-VHLF1/F1 mice undergo Cre mediated excision of the floxed VHL allele in only those cells where the osteocalcin promoter is activated, i.e., osteoblasts (FIG. 1A). These mice developed extremely thick, dense bones with an increase in vascularity. Interestingly, the conditional deletion of HIF-1α in osteoblasts results in thinner bones (data not shown). On specimen collection, the bones in ΔVHL mice were noted to appear blood filled when compared with controls (FIG. 1B). A marked increase in bone volume per total volume in the femur at 6 weeks of age was noted by microCT in ΔVHL mice compared to control littermates (FIG. 1C). The increased vascularity was confirmed by microCT angiography using a technique adapted in collaboration with Dr. Robert Guldberg (Georgia Tech)(Duvall et al., 2004, Am. J. Physiol. Heart Circ. Physiol. 287:H302-10). The animals were euthanized and perfused with a silicon contrast agent, (Microfilm MV-122). The limb was dissected and decalcified, and microCT scanning with 3-D reconstruction of the vasculature was performed. Greatly increased vessel volume within the bone is noted in the ΔVHL mice compared to control littermates (FIG. 1D).

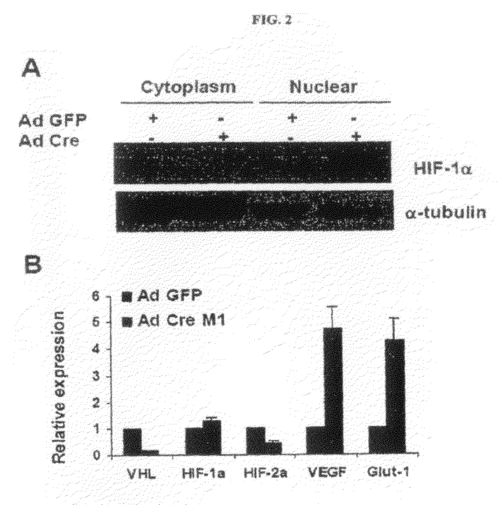

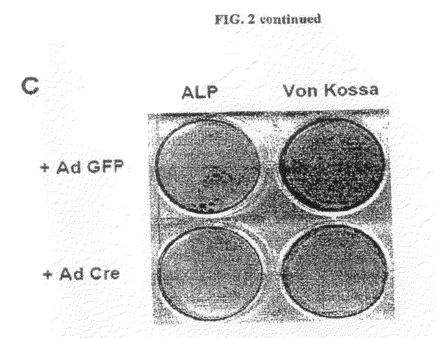

To examine the effect of HIF-1α activation on osteogenesis independent from angiogenesis, primary osteoblasts from the floxed VHL calvaria were infected with adenoviral vectors expressing Cre or GFP as control. Osteoblasts increased levels of HIF-1α in the nucleus in response to VHL deletion compared to control as shown by western blotting (FIG. 2A). Increased expression of downstream targets VEGF and Glut-1 was seen by real time PCR (FIG. 2B). Proliferation was not changed (data not shown) and differentiation as assessed by alkaline phosphatase and Von Kossa staining was similar to controls (FIG. 2C). In addition, overexpression of HIFs does not effect osteoblast proliferation and apoptosis in vitro as determined by FACS analysis (data not shown) or osteoblast differentiation as determined by relative expression of VHL, HIF-2α, Runx2, and osteocalcin (data not shown).

These results indicate that the increased osteogenesis seen in the ΔVHL mice results mainly through cell non-autonomous mechanisms which promote increased angiogenesis. In addition, these results suggest that constitutive activation of the HIF-1α pathway does not grossly disturb osteoblast function, at least in vitro.

### Example 2

### Increased HIF-1α in DO Increases Angiogenesis and Bone Formation

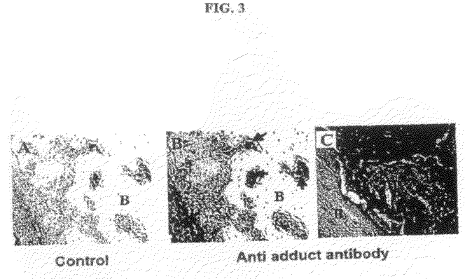

As discussed above, HIF-1α and its downstream targets are strongly induced by distraction in the mouse model. Furthermore, cells within the distraction gap consistent with osteoblasts are hypoxic by staining with pimonidazole (FIG. 3). Pimonidazole, which forms adducts with proteins at pO2 less than 10 mm Hg, was administered to mice immediately before an episode of distraction. The animals were sacrificed 3 hours later, and the bones harvested and sectioned. The pimonidazole-protein adducts were then detected by immunohistochemistry. Intense staining was seen in the distraction gap, and specifically, cells lining areas of new bone formation were noted to be hypoxic (FIG. 3C).

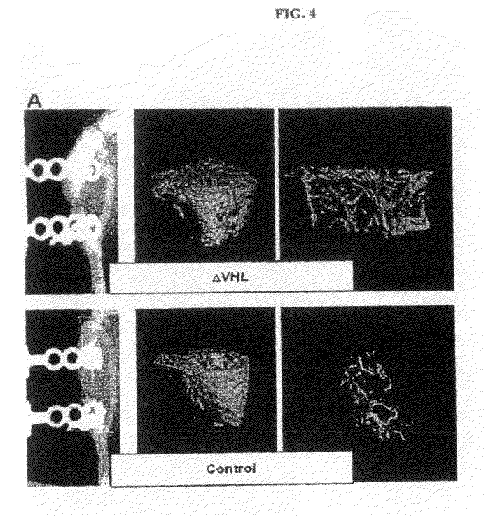

To test whether increased HIF-1α activation over the extent induced by hypoxia within the distraction gap could further increase angiogenesis and bone formation in DO, the procedure was performed in OC-Cre-VHLF1/F1 mice and compared to control VHLF1/F1 mice. In the standard distraction protocol (0.15 mm/day), both the mutant and VHL intact mice healed the distraction gap as noted by x-ray and microCT (FIG. 4A). Quantitation of bone volume per total volume, as assessed by microCT at the end of consolidation showed no difference (FIG. 4B).

To evaluate angiogenesis, the Microfilm silicone contrast perfusion technique was used. Strikingly, the vascularity induced in the distraction gap was observed to be almost doubled in the OC-Cre-VHLF1/F1 mice at the end of the distraction period as measured by vessel volume/total volume of regenerate in the distraction zone (reconstructed vasculature FIG. 4A, quantitation, FIG. 4B). This indicates that despite the dramatic upregulation of angiogenic factors during routine DO, a further Increase in vascularity can be achieved by increased HIF-1α. To test whether bone healing could be improved in a setting where delayed healing was expected, the rate of distraction was doubled (0.3 mm/day). Very little bone healing was seen in the distraction gap of the control mice, whereas the distraction gap is nearly healed in the OC-Cre-VHLF1/F1 mice as shown by x-ray and microCT (FIG. 4C). These findings provide proof of principle that Hi-1α activation can increase angiogenesis and bone formation in DO.

### Example 3

### Pharmacologic Induction of HIF-1α in Osteoblasts by Prolyly Hydroxylase Inhibition

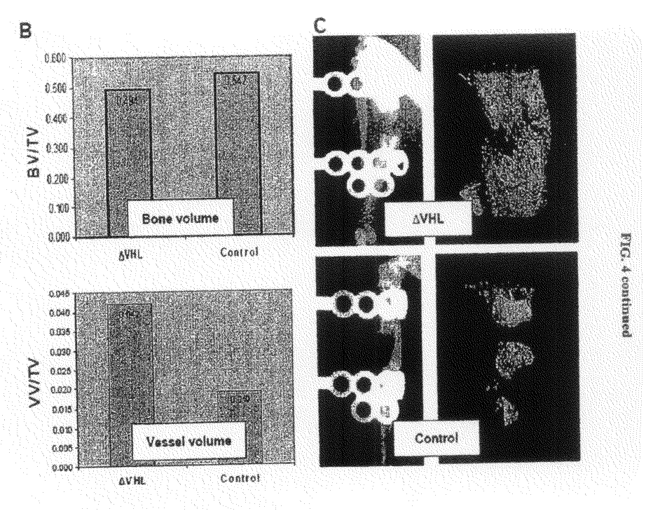

As discussed above, pharmacologic agents that block prolyl hydroxylation of HIF-1α have been studied in a variety of cell lines, animal models, and clinical applications. To confirm the presence of the PHDs in osteoblasts, western blotting of total protein from primary mouse osteoblasts was performed, using primary antibodies to mouse PHD1 and 3 with α-tubulin used as a loading control. Levels of PHDs 1 and 3 were similar in cells with adenoCre mediated VHL excision and control cells with adenoGFP transfection (FIG. 5A). An antibody for mouse PHD2 is not commercially available.

The class of PHDs acting on HIF-1α require oxygen, iron, and 2-oxyglutarate as co-factors. Small molecules that block these enzymes are iron chelators, such as cobalt and desferrioxamine, or 2-oxyglutarate analogs such as L-mimosinc and 3,4 DHB, and DMOG. A schematic of the PHD enzyme and the structures of the 2-oxyglutarate analogs is reproduced in FIG. 5. A number of previous studies have shown an increase of VEGF and other HIF-1α target genes in cells treated with the small molecule PHD inhibitors selected here. Because the effects of the PHD inhibiting agents have not been tested in primary osteoblasts, studies were performed to evaluate their ability to increase HIF-1α. Osteoblasts were grown to near confluence, and then exposed to varying concentrations of cobalt chloride. At time zero, and after 3, 6, 12, and 24 hours of exposure, the cells were harvested and nuclear protein was extracted. Western blotting of nuclear protein for HIF-1α demonstrated a time and dose dependent increase in nuclear HIF-1α when exposed to cobalt.

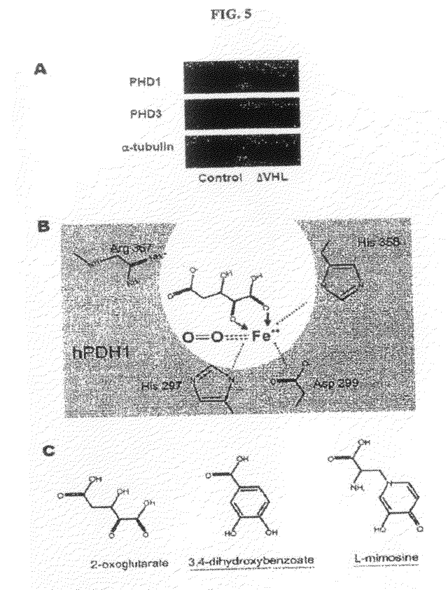

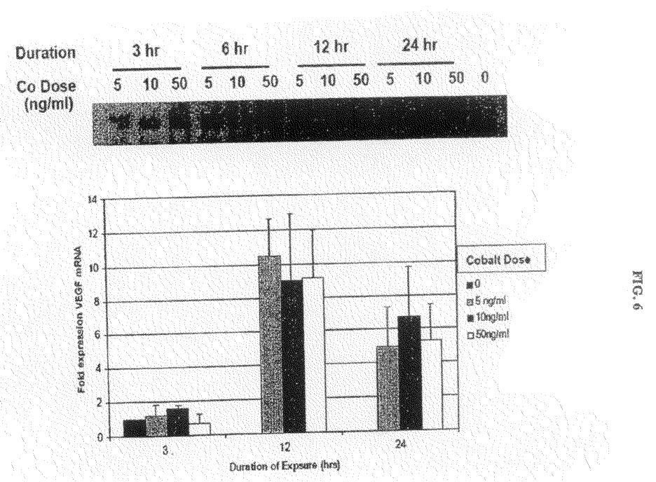

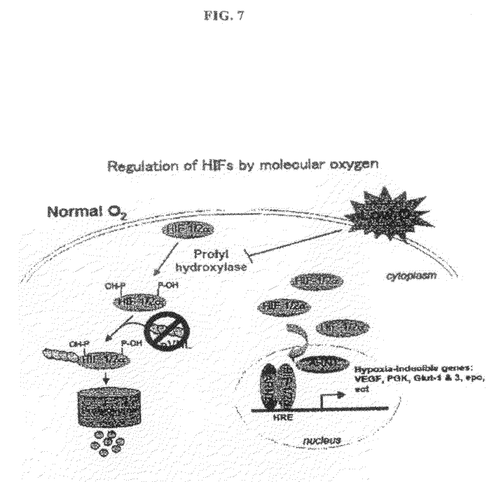

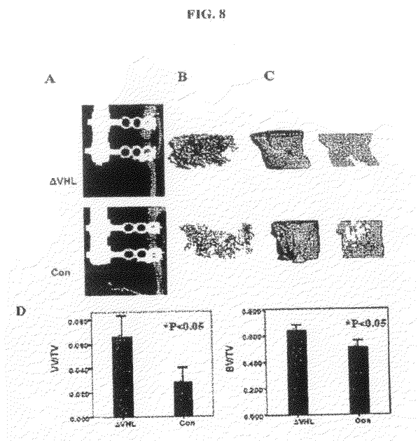

To confirm that a downstream target affecting angiogenesis was upregulated, VEGF mRNA expression was evaluated by real time PCR (FIG. 6). The osteoblasts were exposed to 5, 10, or 50 ng/mL of cobalt chloride. After, 3, 12, or 24 hours, cells were harvested and mRNA was collected. Real time PCR was performed with β-actin as control. Marked upregulation of VEGF expression was seen (FIG. 6B). In situ hybridization experiments also demonstrated that VEGF is upregulated in ΔVHL mice, and VEGF promotes angiogenesis in these mice (data not shown). These results confirm HIF-1α activation and upregulation of VEGF in osteoblasts following exposure to PHD inhibitors in vitro, concordant with published studies in other cell types. Additionally, they lend support to the approach of using VEGF upregulation as assessed by real time PCR as a surrogate for HIF-1α activation in initial screening rather than the more time consuming approach of western blotting for HIF-1α.

### Example 4

### Methods for Identifying Agents that Increase HIF-1α Activity in Osteoblasts In Vitro

The ability of pharmacologic agents to mimic the hypoxic response in osteoblasts in vitro by increasing HIF-1α activation and upregulating downstream targets that induce angiogenesis is tested. Primary osteoblasts are used as a model because this cell type is likely ultimately the target through which hypoxia influences bone healing. Several of these agents have been used in vivo and in vitro previously, although effects in bone and osteoblasts have not been evaluated.

As discussed in the previous Examples, agents are examined for their ability to activate HIF-1α by interfering with prolyl hydroxylases, thereby blocking VHL mediated HIF-1α degradation. Small molecules are used initially, due to the case of preparation and application, relative low cost, and lack of requirement for vectors or carriers as compared to other approaches such as gene therapy or custom peptides. Additional, more complex strategies also may be analyzed. Published in vitro experiments using these agents to mimic hypoxia have been have been performed in cell lines or with tumor cells, but there is no published data on their use in primary osteoblasts. Desferrioxamine, an iron chelator, as well as a variety of 2-oxyglutarate analogs including L-mimosine, 3,4 DHB, and DMOG are tested. Additional small molecules that inhibit prolyl hydroxylases also will be tested.

Genetic inhibition of HIF-1α degradation in osteoblasts demonstrated an increase in HIF-1α and VEGF expression, but little in vitro effect was seen on subsequent differentiation and mineralization (FIG. 2C). Therefore, HIF-1α accumulation and VEGF expression is used as the first level of screening of agents. Because of the potential for interference with collagen metabolism by blocking C4PHD, evaluation of collagen synthesis is the second level of screening. The desired candidate agents strongly block HIF-1α destruction, resulting in a marked increase in HIF-1α accumulation, but lacks major negative effects on collagen synthesis.

For all in vitro screening experiments, primary mouse calvarial osteoblast cultures from wild type FVB/N mice 30-36 hours after birth are used as previously described (Aronson, 1994, Craniofacial Journal 31:473-82). To evaluate activation of HIF-1α and downstream targets in response to the pharmacologic agents, real time PCR is used to evaluate VEGF upregulation as described in the previous Examples, and HIF-1α activation may be confirmed by Western blotting. Osteoblasts are grown to pre-confluence and exposed to a range of doses of the candidate pharmacologic agents to examine the HIF-1α response. Several hours exposure may be required to detect an effect. PBS or another diluent vehicle is used for a negative control. Cells are harvested after 0, 3, 6, 12, and 24 hours of continuous exposure to a range of concentrations of the agents. Using standard methods, mRNA is extracted. Real time PCR is performed to quantify the upregulation VEGF expression with β-actin as a control. This data serve as the first level of screening as it is quantitative, and a large number of samples can be evaluated simultaneously, Western blotting is used to confirm HIF-1α activation by selected agents that have strongly upregulated VEGF expression using the same time course and the most effective doses, Nuclear protein is extracted using a commercially available kit (NE-PER, Pierce). Following gradient gel electrophoresis and membrane transfer, Western blotting is performed using a polygonal antibody to HIF-1α (Novus) as primary antibody. Blots are developed using ECL detection. Membranes are then washed and reprobed for β-actin as a loading control. Films are scanned, for example, by using NIH Image, and the image density of HIF-1α blots is normalized to blots for β-actin of the corresponding samples.

Agents demonstrating strong induction of HIF-1α are then evaluated for possible effects on collagen synthesis. Cells are grown to near confluence. Media is changed, and PHD inhibiting agents or PBS (control) are added. After 24 hours, secreted collagen in the media is measured. Supernatants are precipitated with 10% TCA, and Western blotting is performed for collagen I using a rabbit polyclonal antibody (Abcam, Cambridge, UK). Inage density of the blots is expressed as a percentage of control. Only α1 chains that have undergone proline hydroxylation can join in a procollagen triple helix and be secreted. Therefore, impaired collagen prolyl hydroxylation will be reflected by accompanying decrease in secreted collagen molecules. Additionally, standard [¹⁴C]-proline incorporation assays are performed to assess type I collagen proline hydroxylation.

The described methods of Western blotting for HIF-1α and real time PCR for VEGF expression are standard techniques; however, higher throughput strategies are also contemplated for evaluating additional agents. These strategies may involve, for example, techniques utilizing a 96-well format such as HIF-1α ELISA (commercially available from R&D Systems, Minneapolis, Minn.), or use of stably transfected cell lines with a luciferase reporter linked to a hypoxic response element.

Significant toxicity from short term use of PHD inhibitors has not been observed in vivo, although many PHD inhibitors have been observed to modestly inhibit cell proliferation in vitro. Signs of cell death or diminished proliferation are analyzed during the experiments, and proliferation and apoptosis are assessed by flow cytometry following BrdU or Annexin V treatment, respectively. As mentioned above, overexpression of HIFs does not effect osteoblast proliferation and apoptosis in vitro as determined by FACS analysis (data not shown).

HIF-1α activation through VHL deletion in osteoblasts does not alter proliferation or osteogenic differentiation. In contrast, osteogenesis is markedly increased in vivo in the ΔVHL mice. These results, coupled with the finding of dramatically increased vascularity, indicate that the increased osteogenesis seen in the ΔVHL mice results mainly through cell non-autonomous mechanisms which promote increased angiogenesis. As such, in selecting the best candidate agents for local application, HIF-1α induction will be prioritized over other hi vitro findings.

Concern may arise as to whether osteoblasts, per se, are actually the cells responsible for bone formation in DO, and consequently whether they are appropriate for the in vitro studies. As discussed in the background section, during the latency phase, mesenchymal cells migrate to the injury site. As distraction proceeds, a collagen framework is formed and bone is formed directly on this framework by an intramembranous process. Intramembranous bone formation is generally thought to be osteoblast mediated, and histology in the distraction zone shows cuboidal cells lining areas of new bone formation. These cells are key to osteogenesis and appear and function like osteoblasts. The calvarial osteoblast culture technique is similar to techniques for culturing mesenchymal stem cells from marrow of long bones, and may contain many of the same cell lineages. A distinct advantage is the greater reliability of the calvarial osteoblast cultures. Finally, the ability of manipulation of the osteoblasts to impact bone formation in DO is demonstrated herein by the data showing that VHL deletion in osteoblasts resulted in increased vascularity in response to DO and improved bone volume in healing DO at supraoptimal distraction rates.

### Example 5

### Methods for Identifying Agents that Enhance Bone Formation In Vivo

Small molecules that pharmacologically increase HIF-1α activity as identified in Example 4 are analyzed to determine if they can enhance bone formation in vivo. An osteoblast specific genetic mouse model of increased HIF-1 activation demonstrated that increased angiogenesis through this pathway increases bone formation in vivo. A murine model of DO is used to evaluate the effects of increased HIF-1α on bone formation.

### a) Distraction Osteogenesis

Mature mice (8-12 weeks old) are anesthetized by isoflurane inhalation. A sterile preparation is performed. An incision is performed over the anterolateral left tibia. The bone is exposed taking care to minimize periosteal stripping. A 6 mm track distractor (KLS Martin, Jacksonville, Fla.) is attached to bone with ligature wire. The fibula and tibia are then osteotomized and reapproximated, if necessary. A mini-osmotic pump (Alzet, Cupertino, Calif.) is filled with the agent of interest and implanted subcutaneously on the back. The tubing is routed to the osteotomy site. The pump is designed to give a constant infusion at a rate which can vary from 0.25-10 μL/hr based on the model chosen. The desired dosing can be achieved by varying the concentration. The wounds are closed with absorbable monofilament suture. An X-ray is used to confirm alignment. Following a latency period of five days, distraction commences. Distraction is achieved by turning a screw on the device (one turn equals 0.3 mm distraction). In the standard protocol, active distraction is for a period of 10 days with ½ turn (0.15 mm) per day, for a total lengthening of 1.5 mm, or approximately 8% of the original tibial length. The consolidation phase is then a further 14 days. For the accelerated lengthening protocol, active distraction is lengthened 0.3 mm/day for 10 days (3 mm), or approximately 15% of total length.

Histologic, radiographic, and biomechanical evaluation of healing is performed. Histologic examination of the distracted bone is performed at the end of latency, during distraction, at the end of distraction, and during consolidation. Specimens are stripped of soft tissue, fixed in 4% paraformaldehyde, and decalcified in EDTA. Specimens are embedded in paraffin, sectioned, and mounted onto slides. Examination qualitatively evaluates described features of DO including fibrous tissue, osteoblasts, vessel ingrowth, and bone and cartilage formation. Quantitative analysis of bone formation is performed by color match analysis of Safranin-O stained sections to yield a percent of the distraction gap occupied by bone, cartilage, or other tissue. Immunohistochemistry is performed for HIF-1α and VEGF using standard techniques. Briefly, sections are treated with hydrogen peroxide to block endogenous peroxidase, blocked with goat serum, and incubated with commercially available primary antibodies. After rinsing with PBS, secondary biotinylated anti-rabbit antibody is applied, and the slides are incubated with 3,3-diaminobenzidine tetrachloride solution (DAB). Counterstain with hematoxylin are applied.

Radiographic evaluation includes, for example, Digital x-rays, microCT, and microCT angiography. Digitally captured x-ray images are performed using a Faxitron MX-20 benchtop machine following surgery to confirm alignment. Repeat x-rays are used to assess bone formation on day 7 (end of consolidation), day 14 (during distraction), and after 7 and 14 days of consolidation. Micro computed tomography is performed to further evaluate bone healing at consolidation days 7 and 14. Mice are sacrificed and the limbs dissected and the fixators removed. A high resolution (8-36 μm voxel size) micro-T imaging system is utilized (μCT 40, Scanco Medical, Bassersdorff, Switzerland). Approximately 300 contiguous axial slices are obtained at 70 kV and 112 μA with a voxel size of 16 μm. The region of interest is defined as inclusive of the slices between the most proximal and distal slices containing 50% of the original bone cortex. A bone segmentation threshold setting of 235 (segmentation 1.2/2/235) is employed. Direct calculation of morphometric parameters are obtained with the accompanying software. Bone volume/total volume is the primary microCT endpoint.

To evaluate angiogenesis, a perfusion micro-CT technique is used at the completion of distraction and after 7 and 14 days of consolidation. The technique has been adapted to evaluate vasculature within bone. The animals are euthanized, the vasculature flushed with heparinized saline, then fixed with formalin via a needle inserted in the left ventricle. A silicone rubber compound containing lead chromate (Microfil® MV-122, Flow Tech, Carver, Mass.) is then injected until the vasculature is observed to be perfused (turns yellow). After polymerization, the limbs are dissected and the bones are decalcified. MicroCt analysis is then performed. The volume of interest is defined as above. A threshold of 306 (segmentation 1.2/2/306) is used, based on visual interpretation of thresholded 2-D tomograms in specimens from preliminary evidence. Three dimensional histomorphometric values vessel volume, connectivity, number, thickness, thickness distribution, separation, and degree of anisotropy are calculated with the accompanying software. Vessel volume as a percent of total volume of the region of interest (distraction gap) is the primary endpoint assessed.

In order to ensure that the increased bone that is formed following induction of the HIF-1α pathway is biomechanically sound, the structural properties of the regenerate at both gross and microstructural levels are investigated. Mechanical integrity is expected to correlate with BV/TV by microCT. Evaluations of the distracted bones are also performed after 7 and 14 days of consolidation. Bending has been cited as primary failure method for bone following DO. The bones are therefore tested to failure by three-point bending tests to measure structural and material properties at a whole bone level, and then nanoindentation is done to evaluate the material properties at a microstructural level. Following distraction, the animals are euthanized, and the tibias are disarticulated and excised for mechanical testing. The specimens are stored in saline. Destructive testing is done on a custom made three-point bending fixture with a support span of 12 mm using an 858 MiniBonix Materials Testing System (MTS Systems, Eden Prairie, Minn., USA). The specimens are flexed about their medio-lateral axis and loaded at the rate of 0.03 mm/s. Stiff-ness, peak load, and energy to failure are obtained from the load-displacement graphs while elastic modulus (E) and ultimate strength (σf) are obtained incorporating the cross-sectional geometry measures from microCT. Nanoindentation is performed to evaluate microstructural properties. Sections are dehydrated, embedded, and sectioned, then ground, polished, and cleansed ultrasonically. Depth-control nanoindentation tests are conducted using a Nanoindenter XP (MTS Systems, Oak Ridge, Tenn.) with a Berkovich diamond indenter. The loading/unloading process consists of loading to a maximum indentation depth of 500 nm at a strain rate of 0.05 l/s with a 100 s hold period at maximum load to minimize viscoelasticity effects and 200 s hold period at 10% of the maximum load during unloading to correct the displacement due to thermal drift. The modulus and hardness are calculated from the load-displacement curves using Oliver and Pharr method.

Based on published literature and the present disclosure, increased HIF-1α activation in osteoblasts during development and during skeletal repair in DO leads to dramatically increased bone formation and vascularity in vivo. Pharmacologic agents can increase HIF-1α and downstream target VEGF levels in osteoblasts in vitro. Therefore, it is expected that increased HIF-1α activation by local application of pharmacologic agents will improve healing of DO by increasing angiogenesis and bone formation in the distraction gap. Primary endpoints are BV/TV by microCT for bone healing and VV/TV by microCT for angiogenesis. A significant increase in VV/TV in the treated group in both the standard and accelerated protocols is anticipated. As discussed previously, standard distraction ultimately leads to routine bone healing without intervention. However, BV/TV may differ at the midpoint of consolidation, indicating faster healing, or following rapid distraction, indicating ability to heal larger gaps in the same time. Either result could translate to decreased total time of external fixation required per length obtained (analogous to bone healing index clinically). Histology will provide additional data on bone formation, vascularity, and HIF-1α activation.

Both cobalt and desferrioxamine have been shown in vivo and In vitro to mimic hypoxia and induce HIF-1α. Additionally, prolonged administration of desferrioxamine in patients with thalassemia has been shown to lead to a bone phenotype, confirming that this class of agents can affect bone. The mechanism of the observed dysplasia is not known. In high doses, the agents may inhibit cell cycle progression, and the effects could in fact be deleterious on an anabolic process such as bone formation in contrast to ischemia models in which the agents have more commonly been used. Animal models using desferrioxamine to induce local vascular ingrowth suggest that local toxicity is not prohibitive. The doses and route proposed represent a starting point based on published series to maximize effect while attempting to avoid toxicity. Additionally, use of a vehicle for local administration may be required, though a single local injection (fibroblast growth factor) has been effective in improving healing in a rabbit distraction model. If necessary, fibrin gel has been successfully used in other models and could be easily adapted to this protocol. The described delivery system, however, allows sustained application of the desired agents which should in theory serve to maximize effect and minimize potential toxicity. It has been used successfully to deliver IGF-1 to improve intramembranous healing of calvarial defects in a rat model. Control animals with vehicle administration alone are included. Additional issues which may require altering the experimental protocols include optimizing dosage and administration.

Activation of the HIF-1α pathway is likely to increase angiogenesis and osteogenesis, at least in part, by increasing VEGF. Bone healing was improved by local application of VEGF in a rabbit fracture non-union model (Eckardt et al., 2005, J Bone Joint Surg. Br, 87:1434-38). However, a similar investigation of local application of VEGF or VEGF inhibitor to distraction sites did not show improved or impaired bone formation during DO in rabbits. Those authors speculated one possible explanation for the lack of improvement in bone formation in distraction could be near maximal stimulation by distraction alone, or lack of additional vascular promoters such as angiopoetin. HIF-1α impinges on a number of angiogenic factors in addition to VEGF, so it is reasonable to expect that activating the HIF-1α pathway may yield different results. As mentioned in the background section, in a non-union model, factors in addition to vascular formation are required. Since HIF-1α is known to not only be important in angiogenesis, but also involved in cell recruitment and inflammation, and impinges on mesynchymal stem cell differentiation, it is uniquely qualified to muster the necessary resources for skeletal repair.

OC-Cre-VHL mice exhibit greatly increased bone formation, but the bone is immature, woven bone. Therefore, it may be advantageous to produce a large quantity of bone in the distraction and early consolidation period, and then remove the stimulus to allow HIF-1α activity to normalize to allow normal remodeling and maturation of the bone in the distraction gap. Accordingly, the design of the osmotic pumps lends itself most easily to delivery for a two week time course, coinciding with the latency and distraction phases. Alternately, therapy could be initiated by waiting to fill the pumps at the beginning of the distraction phase. Replacing the pumps may be necessary if a longer duration of treatment is required. The ability to vary the timing and the duration of intervention is a potential advantage over gene therapy approaches.

We claim:

1. A method of identifying a compound that improves bone mass or volume, wherein the method comprises the steps of:
a. providing an osteoblast or bone precursor cell;
b. contacting the cell with a test compound; and
c. determining whether an increase in dimerization, nuclear translocation, and/or transcriptional activity of a HIF-1α/HIF-1β complex occurs in the cell contacted with the compound, said increase being an indication that the compound improves bone mass or volume.

2. The method of claim 1, wherein the transcriptional activity of a HIF-1α/HIF-1β complex is determined by measuring the transcription activation of a HIF-1α target gene selected from the group consisting of VEGF; nitric oxide synthase 2; heme oxygenase 2; α_{1B}-adrenergic receptor; erythropoietin; transferrin; ceruloplasmin; transferrin receptor; cyclin G2; p21; IGF-2; IGF-binding protein 1, 2 and 3; glucose transporter 1, and 3; hexokinase 1, and 2; phosphofructokinase L; pyruvate kinase M; a HIF-prolyl hydroxylase; and carbonic anhydrase.

3. The method of claim 1, wherein the transcriptional activity of the HIF-1α/HIF-1β complex is determined by measuring the transcription activation of VEGF or Glut-1.

4. The method of claim 2, wherein the transcription activation is measured by real-time PCR.

5. The method of claim 1, wherein the expression level of HIF-1α protein is measured.

6. The method of claim 5, wherein the expression level is measured by Western blotting.

7. The method of claim 1, wherein the method comprises an additional step:
d. determining whether an increase or decrease in collagen synthesis occurs in the cell contacted with the compound.

8. The method of claim 7, wherein the increase or decrease in collagen synthesis is detected by measuring the level of secreted collagen in the media.

9. The method of claim 8, wherein the level of secreted collagen is measured by Western blotting.

10. A process for making a compound that increases osteoblast cell differentiation, improves bone mass or volume, and/or promotes osteogenesis, comprising the steps of:
a. providing an osteoblast or bone precursor cell;
b. contacting the cell with a test compound;
c. determining whether an increase in dimerization, nuclear translocation, and/or transcriptional activity of a HIF-1α/HIF-1β complex occurs in the cell contacted with the compound, said increase being an indication that the compound increases osteoblasts cell differentiation, improves bone mass or volume, and/or promotes osteogenesis; and
d. manufacturing the compound.

11. A method of identifying a compound that promotes osteogenesis, wherein the method comprises the comprises the steps of:
a. providing an osteoblast or bone precursor cell;
b. contacting the cell with a test compound; and
c. determining whether an increase in dimerization, nuclear translocation, and/or transcriptional activity of a HIF-1α/HIF-1β complex occurs in the cell contacted with the compound, said increase being an indication that the compound promotes osteogenesis.

12. The method of claim 11, wherein the compound promotes osteogenesis by one or more of increasing osteoblast formation, increasing osteoid volume, decreasing osteoclast formation, and decreasing osteoclast function.

13. The method of claim 11, wherein the transcriptional activity of a HIF-1α/HIF-1β complex is determined by measuring the transcription activation of a HIF-1α target gene selected from the group consisting of VEGF; nitric oxide synthase 2; heme oxygenase 2; α_{1B}-adrenergic receptor; erythropoietin; transferrin; ceruloplasmin; transferrin receptor; cyclin G2; p21; IGF-2; IGF-binding protein 1, 2 and 3; glucose transporter I (Glut-1), and 3; hexokinase 1, and 2; phosphofructokinase L; pyruvate kinase M; a HIF-prolyl hydroxylase; and carbonic anhydrase.

14. The method of claim 11, wherein the transcriptional activity of a HIF-1α/HIF-1β complex is determined by measuring the transcription activation of VEGF or Glut-1.

15. The method of claim 14, wherein the transcription activation is measured by real-time PCR.

16. The method of claim 11, wherein the expression level of HIF-1α protein is measured.

17. The method of claim 16, wherein the expression is measured by Western blotting.

18. The method of claim 11, wherein the method comprises an additional step:
d. determining whether an increase or decrease in collagen synthesis occurs in the cell contacted with the compound.

19. The method of claim 18, wherein the increase or decrease in collagen synthesis is detected by measuring the level of secreted collagen in the media.

20. The method of claim 19, wherein the level of secreted collagen is measured by Western blotting.

21. A method of identifying a compound that increases osteoblast cell differentiation, wherein the method comprises the steps of:
a. providing an osteoblast or bone precursor cell;
b. contacting the cell with a test compound; and
c. determining whether an increase in dimerization, nuclear translocation, and/or transcriptional activity of an HIF-1α/HIF-1β complex occurs in the cell contacted with the compound, said increase being an indication that the compound increases osteoblast cell differentiation.

22. The method of claim 21, wherein the transcriptional activity of a HIF-1α/HIF-1β complex is determined by measuring the transcription activation of a HIF-1α target gene selected from the group consisting of VEGF; nitric oxide synthase 2; heme oxygenase 2; α_{1B}-adrenergic receptor; erythropoietin; transferrin; ceruloplasmin; transferrin receptor; cyclin G2; p21; IGF-2; IGF-binding protein 3, 2 and 3; glucose transporter 1, and 3; hexokinase 1, and 2; phosphofructokinase L; pyruvate kinase M; a HIF-prolyl hydroxylase; and carbonic anhydrase.

23. The method of claim 21, wherein the transcriptional activity of tie HIF-1α/HIF-1β complex is determined by measuring the transcription activation of VEGF or Glut-1.

24. The method of claim 22, wherein the transcription activation is measured by real-time PCR.

25- The method of claim 21, wherein the expression level of HIF-1α protein is measured.

26. The method of claim 25, wherein the expression level is measured by Western blotting.

27. The method of claim 21, wherein the method comprises an additional step;
d. determining whether an increase or decrease in collagen synthesis occurs in the cell contacted with the compound.

28. The method of claim 27, wherein the increase or decrease in collagen synthesis is detected by measuring the level of secreted collagen in the media.

29. The method of claim 28, wherein the level of secreted collagen is measured by Western blotting.

30. The present invention also provides for methods of identifying a compound capable of decreasing osteoblast cell differentiation, decreasing bone mass or volume, and/or increasing osteoclast cell differentiation, wherein the methods
a. providing an osteoblast or bone precursor cell;
b. contacting the cell with a test compound; and
c. determining whether a decrease in dimerization, nuclear translocation, and/or transcriptional activity of a HIF-1α/HIF-1β complex occurs in the cell contacted with the compound, said decrease being an indication that the compound decreases osteoblast cell differentiation, decreases bone mass or volume, and/or increases osteoclast cell differentiation.

31. A process for making a compound that decreases osteoblast cell differentiation, decreases bone mass or volume, and/or increases osteoclast cell differentiation, comprising the steps of:
a. providing an osteoblast or bone precursor cell;
b. contacting the cell with a test compound; and
c. determining whether a decrease in dimerization, nuclear translocation, and/or transcriptional activity of a HIF-1α/HIF-1β complex occurs in the cell contacted with the compound, said decrease being an indication that the compound decreases osteoblast cell differentiation, decreases bone mass or volume, and/or increases osteoclast cell differentiation; and
d. manufacturing the compound.

32. A compound for use in promoting the activity of HIF-1α for the treatment of a disease state, such as, but not limited to osteoporosis, osteopenia, or a bone-related disorder selected from the group consisting of osteoporosis, bone fractures, hypercalcemia of malignancy, osteopenia or osteolytic lesions due to bone metastases, periprosthetic osteolysis, familial expansile osteolysis, periodontal disease, tooth loss, rheumatoid arthritis, osteoarthritis, hyperparathyroidism, Paget's disease, osteodystrophy, myositis ossificans, Bechterew's disease, malignant hypercalcernia, bone loss, bone abnormalities due to steroid hormone treatment, bone abnormalities caused by cancer therapeutics, abnormally increased bone turnover, osteomalacia, Bechet's disease, hyperostosis, osteopetrosis, osteogenesis imperfecta, rachitis, immobilization-induced osteopenia, expansile skeletal hyperphosphatasia, and glucocorticoid-induced osteoporosis.

33. A method for strengthening a bone graft, inducing vertebral synostosis, enhancing long bone extension, the treatment and promotion of healing of bone fractures and osteotomies, enhancing bone healing following facial reconstruction, maxillary reconstruction, and/or mandibular reconstruction in a vertebrate, comprising tie step of:
a. administering to said vertebrate a composition comprising a therapeutically effective amount of a compound of claim 32, a prodrug, or a pharmaceutically acceptable salt thereof, or a stereoisomer or diastereomeric mixture of said compound, prodrug, or salt.

34. The method of claim 33, wherein the composition is administered locally to the site of bone reconstruction.

35. The method of claim 33, wherein the composition is administered systemically.
